(19) United States
(12) Patent Application Publication
(10) Pub. No.: US 2025/0204999 A1
(43) Pub. Date: Jun. 26, 2025

(54) SYSTEMS AND METHODS OF SURGICAL IMAGE GUIDANCE

(71) Applicant: Vanderbilt University, Nashville, TN (US)

(72) Inventors: Michael I. Miga, Nashville, TN (US); Ingrid M. Meszoely, Nashville, TN (US)

(21) Appl. No.: 18/989,775

(22) Filed: Dec. 20, 2024

Related U.S. Application Data
(60) Provisional application No. 63/613,891, filed on Dec. 22, 2023.

(51) Int. Cl. A61B 34/32; A61B 34/20; A61B 90/00

(52) U.S. Cl. CPC A61B 34/32; A61B 2034/2057; A61B 2034/2063; A61B 2090/367; A61B 2090/374

(57) ABSTRACT

A surgical image guidance system, including: a processing circuit including a processor and memory, the memory having instructions stored thereon that, when executed by the processor, cause the processing circuit to: receive, from an optical device, surface data characterizing a surface of a portion of soft tissue; receive, from an intraoperative imaging device, subsurface data characterizing a subsurface of the portion of soft tissue, wherein the subsurface includes a region of interest; measure, based on the surface data, deformation of the surface of the portion of soft tissue; update, based on at least one of (i) the deformation of the surface and (ii) the subsurface data, a model including a representation of the region of interest; and transmit a visual representation of the model to be displayed to a user.

### CROSS-REFERENCE TO RELATED APPLICATIONS

This application claims the benefit of U.S. Provisional Patent Application No. 63/613,891, filed on Dec. 22, 2023, the entire contents of which are incorporated herein by reference.

### STATEMENT REGARDING FEDERALLY SPONSORED RESEARCH OR DEVELOPMENT

This invention was made with government support under EB022380 awarded by the National Institutes of Health. The government has certain rights in the invention.

### BACKGROUND

The present disclosure relates generally to the field of surgical guidance, and more specifically to systems and methods of surgical image guidance.

### SUMMARY

In some aspects, the techniques described herein relate to a surgical image guidance system, including: a processing circuit including a processor and memory, the memory having instructions stored thereon that, when executed by the processor, cause the processing circuit to: receive, from an optical device, surface data characterizing a surface of a portion of soft tissue; receive, from an intraoperative imaging device, subsurface data characterizing a subsurface of the portion of soft tissue, wherein the subsurface includes a region of interest; measure, based on the surface data, deformation of the surface of the portion of soft tissue; update, based on at least one of (i) the deformation of the surface and (ii) the subsurface data, a model including a representation of the region of interest; and transmit a visual representation of the model to be displayed to a user.

In some aspects, the techniques described herein relate to a surgical image guidance system, wherein the intraoperative imaging device includes an ultrasound image device. In some aspects, the techniques described herein relate to a surgical image guidance system, wherein the optical device includes a camera. In some aspects, the techniques described herein relate to a surgical image guidance system, wherein the camera includes a stereo camera. In some aspects, the techniques described herein relate to a surgical image guidance system, wherein the optical device includes an optical tracker. In some aspects, the techniques described herein relate to a surgical image guidance system, wherein the instructions further cause the processing circuit to: track a position of a surgical instrument; and display the position of the surgical instrument on the visual representation of the model. In some aspects, the techniques described herein relate to a surgical image guidance system, wherein the region of interest includes soft tissue to be removed, and wherein the visual representation of the model includes an illustration of a surgical margin associated with the soft tissue to be removed. In some aspects, the techniques described herein relate to a surgical image guidance system, wherein updating the model includes updating the representation of the region of interest to reflect deformation of the subsurface of the portion of soft tissue. In some aspects, the techniques described herein relate to a surgical image guidance system, wherein the instructions further cause the processing circuit to: receive imaging data characterizing the subsurface of the of the portion of soft tissue; and generate, based on the imaging data, the model including the representation of the region of interest. In some aspects, the techniques described herein relate to a surgical image guidance system, wherein the imaging data includes magnetic resonance imaging (MRI) data.

In some aspects, the techniques described herein relate to a surgical assistance system, including: a robotic arm assembly having an optical device coupled thereto; and a processing circuit including a processor and memory, the memory having instructions stored thereon that, when executed by the processor, cause the processing circuit to: autonomously control movement of the robotic arm assembly to position the optical device such that the optical device has at least a partial view of (i) a portion of soft tissue under operation or (ii) a fiducial associated with the portion of soft tissue; receive, from the optical device, surface data characterizing a surface of the portion of soft tissue under operation; determine, based on the (i) surface data and (ii) intraoperative ultrasound data, deformation of a subsurface of the portion of soft tissue under operation; and cause a visual representation of the deformation of the subsurface to be displayed to a user; and wherein the visual representation includes an indication of (i) diseased soft tissue or (ii) a region of interest.

In some aspects, the techniques described herein relate to a surgical assistance system, wherein the instructions further cause the processing circuit to: track a position of at least one of (i) an object or (ii) an entity in 3D space; and wherein autonomously controlling movement of the robotic arm assembly includes controlling movement of the robotic arm assembly based on the position of the object or entity in 3D space. In some aspects, the techniques described herein relate to a surgical assistance system, wherein the optical device includes at least one of an optical tracking device or a stereo camera. In some aspects, the techniques described herein relate to a surgical assistance system, wherein determining deformation of the subsurface includes updating a model of the subsurface generated based on magnetic resonance imaging (MRI) data.

In some aspects, the techniques described herein relate to a method of providing surgical image guidance, including: receiving preoperative imaging data of at least a portion of a subject; identifying, based on the preoperative imaging data, an initial location of a lesion; receiving intraoperative imaging data of the portion of the subject; identifying a subsequent location of the lesion by determining, based on the intraoperative imaging data, soft tissue deformation of the portion of the subject; and displaying, to a user, the subsequent location of the lesion.

In some aspects, the techniques described herein relate to a method, wherein determining soft tissue deformation includes: aligning the preoperative imaging data with the intraoperative imaging data using a fiducial to form a composite; and updating a 3D volumetric model of the portion of the subject based on the composite. In some aspects, the techniques described herein relate to a method, wherein the intraoperative imaging data includes at least one of (i) image data from a stereo camera or (ii) subsurface data from an ultrasound device. In some aspects, the techniques described herein relate to a method, wherein the method further includes: tracking a position of a surgical instrument; and wherein displaying the subsequent location of the lesion includes displaying the position of the surgical instrument relative to the subsequent location of the lesion. In some aspects, the techniques described herein relate to a method, wherein displaying the subsequent location of the lesion includes displaying a 3D model of the lesion and a visual representation of the surgical instrument. In some aspects, the techniques described herein relate to a method, wherein the method further includes controlling a position of a robotic arm assembly based on the intraoperative imaging data, and wherein controlling the position of the robotic arm assembly includes adjusting a field of view of an imaging device such that the field of view at least partially includes the portion of the subject.

### BRIEF DESCRIPTION OF THE DRAWINGS

The above and other aspects and features of the present disclosure will become more apparent to those skilled in the art from the following detailed description of the example embodiments with reference to the accompanying drawings.

FIG. 1 is an illustration of a surgical image guidance system deployed in a surgical context, according to an exemplary embodiment.

FIG. 2 is an illustration of a composite display, according to an exemplary embodiment.

FIG. 3 is an illustration of a surgical guidance display, according to an exemplary embodiment.

FIG. 4 is an illustration of a graphical user interface, according to an exemplary embodiment.

FIG. 5 is a flow diagram illustrating a method of positioning a robotic arm assembly, according to an exemplary embodiment.

FIG. 6 is a flow diagram illustrating a method of surgical image guidance, according to an exemplary embodiment.

FIG. 7 is a block diagram of a surgical assistance system, according to an exemplary embodiment.

### DETAILED DESCRIPTION

Referring generally to the FIGURES, described herein are systems and methods of surgical image guidance. Care providers (e.g., surgeons, nurses, etc.) may interact with soft tissue during surgery. For example, a surgeon may make an incision in the soft tissue of a subject in order to remove a subsurface lesion. Soft tissue may deform as result of interaction. For example, a surgeon may press a scalpel to the skin of a subject and the pressure of the scalpel may cause subsurface portions of the soft tissue to move and/or change shape. In many contexts, it is beneficial to locate a position of a region/structure of interest during surgery. For example, a surgeon may want to locate vasculature to avoid when making incisions or may want to locate a subsurface lesion to remove. However, it may be difficult to locate a region of interest because of soft tissue deformation. For example, a surgeon may use medical imaging to identify a location of a lesion to remove, but the lesion may change locations during extraction due to soft tissue deformation caused by the extraction process. Systems and methods of the present disclosure facilitate surgical image guidance to determine a location of a region of interest during surgery and display the location to a care provider (e.g., surgeon, nurse, etc.).

Systems and methods of the present disclosure may offer benefits over conventional systems. For example, conventional systems (e.g., marker-implant systems, guidewire systems, etc.) may be unable to determine the spatial extent of a region/structure of interest (e.g., the 3D volume of a lesion, etc.). The surgical image guidance system of the present disclosure may determine the spatial extent of a region/structure of interest (e.g., a lesion), may provide a visualization of a surgical margin associated with the region/structure of interest, and/or may account for deformation in the region/structure of interest. As another example, conventional systems may be unable to track changes in the spatial extent of a region/structure of interest during surgery. For example, a surgeon using a conventional system may determine a preoperative location of a lesion but may be unable to determine how the lesion moves and/or changes shape as a result of soft tissue deformation during surgery. Moreover, conventional systems and methods of intraoperative imaging may not be well-suited to determining the spatial extent of a region/structure of interest in real time. For example, tumors may not be visible by ultrasound (e.g., because the tumor is small, diffuse, and/or embedded in dense tissue, etc.). The surgical image guidance system of the present disclosure may track the spatial extent of a region/structure of interest in real time, may account for changes in position and/or shape as a result of soft tissue deformation, and may display real time position and/or shape information to a care provider. As another example, in many contexts it may be desirable to verify/validate the outcome of a surgical resection/treatment extent. For example, a surgeon may remove a lesion and it may be desirable to determine that the lesion has been completely removed (e.g., that the surgeon didn't miss a portion of the lesion, etc.). In many contexts, verifying/validation the surgical resection/treatment extent includes verifying/validating a surgical margin associated with a region/structure of interest. For example, a surgeon may verify that two centimeters of normal tissue surround an extracted piece of diseased tissue. The surgical image guidance system of the present disclosure may automate verification/validation by tracking a position/location of a surgical instrument and/or a region/structure of interest in real time while accounting for soft tissue deformation. In various embodiments, the systems and methods of the present disclosure improve upon conventional systems and methods by reducing reoperation rates, reducing the length of operation, and/or increasing surgical accuracy. Reducing reoperation rates may speed up recovery (e.g., because reoperation may delay subsequent therapies, etc.) and/or may improve the treatment experience (e.g., because reoperation may be associated with negative psychological effects, etc.). Reducing the length of operation may save costs. Increasing surgical accuracy may (i) reduce surgical margins such that surgeons do not have to remove as much tissue, (ii) enable less invasive operations such as a lumpectomy rather than a mastectomy, (iii) increase the safety of surgery by helping surgeons navigate sensitive areas such as areas with major vasculature, and/or (iv) facilitate therapeutic techniques such as microwave ablation and/or laparoscopic surgery.

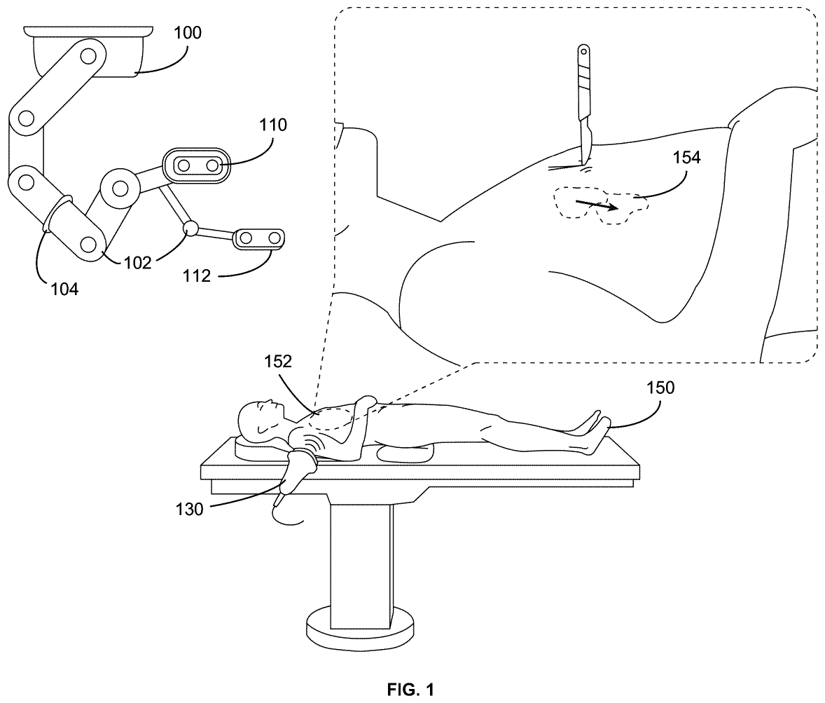

Turning now to FIG. 1, an illustration of a surgical image guidance system deployed in a surgical context is shown, according to an exemplary embodiment. In various embodiments, the surgical image guidance system tracks a position/location of a region/structure of interest and displays the position/location to a user (e.g., a care provider such as a surgeon, nurse, etc.). In various embodiments, the region/structure of interest includes a lesion such as a tumor. Additionally or alternatively, the region/structure of interest may be and/or include other features such as vasculature, a foreign object, skeletal features, a reference (e.g., a marker/guide, etc.), an area undergoing surgery, a planned surgical/interventional path, a portion of soft tissue, and/or the like. A region/structure of interest may be and/or include a surface and/or a subsurface. For example, a region/structure of interest may include a portion of a surface of a subject's skin and subsurface soft tissue corresponding to the portion of the surface. As described herein, tracking a position/location of the region/structure of interest includes tracking a 3D volume. For example, the surgical image guidance system may track a 3D volume representing a lesion and may update the 3D volume in response to changes due to soft tissue deformation (e.g., to deform the 3D volume, etc.). Additionally or alternatively, tracking a position/location may include tracking a discrete point in 3D space.

As shown, surgical image guidance system 100 may monitor a surgery using one or more sensors (shown as optical device 110 and optical tracking device 112). Optical device 110 may be and/or include any optical device such as a camera, a projector, a laser, an optical fiber, a spectrometer, and/or the like. In various embodiments, optical device 110 is and/or includes a stereo camera. Optical tracking device 112 may be and/or include any optical device capable of tracking a physical point in space. For example, optical tracking device 112 may include a camera configured to capture image data of a point in space and software configured to track the point in space across different images. In various embodiments, optical tracking device 112 is configured to track a 3D position/orientation of one or more objects (e.g., surgical instruments, etc.). In various embodiments, surgical image guidance system 100 positions optical device 110 and/or optical tracking device 112 using one or more robotic arm assemblies (shown as robotic arm assemblies 102). In various embodiments, optical device 110 and/or optical tracking device 112 are coupled to robotic arm assemblies 102. Robotic arm assemblies 102 may position the one or more sensors in 3D space. Each robotic arm assembly of robotic arm assemblies 102 may be configured to operate in one or more degrees of freedom. For example, a robotic arm assembly of robotic arm assemblies 102 may be configured to operate in 7 degrees of freedom. In various embodiments, surgical image guidance system 100 may position the one or more sensors independently of one another. For example, surgical image guidance system 100 may position optical device 110 in a first location having a first field of view and may position optical tracking device 112 in a second location having a second field of view. Robotic arm assemblies 102 may include one or more sensors (shown as sensor 104). Sensor 104 may be and/or include a proximity sensor, a contact sensor, and/or the like. In various embodiments, surgical image guidance system 100 receives input from sensor 104 and uses the input to control robotic arm assemblies 102. For example, sensor 104 may sense a person and surgical image guidance system 100 may operate robotic arm assemblies 102 to avoid contacting and/or obstructing the person. Robotic arm assemblies 102 may be positioned in a fixed location and/or non-fixed location. For example, robotic arm assemblies 102 may be positioned on a ceiling of an operating room, on a movable cart, on a patient's bed, on a wall, and/or the like.

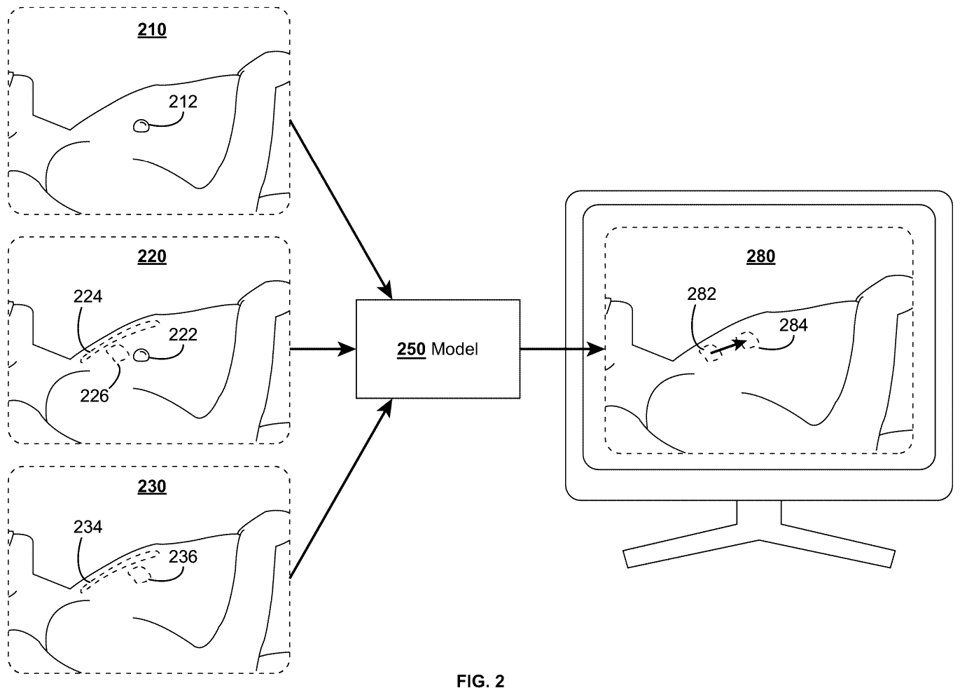

In various embodiments, surgical image guidance system 100 monitors an area associated with a surgical procedure (shown as area 152) performed a subject (shown as subject 150). For example, subject 150 may be undergoing a mastectomy and surgical image guidance system 100 may monitor a chest region of subject 150. In various embodiments, surgical image guidance system 100 monitors the area using the one or more sensors (i.e., optical device 110 and/or optical tracking device 112). For example, surgical image guidance system 100 may generate a 3D point cloud of a surface of area 152 being operated on using optical device 110. Additionally or alternatively, surgical image guidance system 100 may receive one or more inputs to facilitate monitoring area 152. For example, surgical image guidance system 100 may receive subsurface data from an intraoperative imaging device (shown as ultrasound device 130) to characterize a subsurface of area 152. The subsurface data may be and/or include imaging data describing the anatomy of subject 150 (e.g., data from an ultrasound device, an electromagnetic imaging device, etc.). In some embodiments, area 152 is and/or includes a region/structure of interest (shown as region 154). For example, area 152 may correspond to a portion of skin that a surgeon is cutting through to access a subsurface tumor. In various embodiments, surgical image guidance system 100 tracks a position/location of region 154. For example, surgical image guidance system 100 may combine preoperative magnetic resonance imaging (MRI) data, intraoperative ultrasound data, and/or intraoperative image data (e.g., from optical device 110) to track deformation of region 154 during a surgery and/or display a real time position/location of region 154 to a user (e.g., a surgeon, a nurse, etc.). Additionally or alternatively, surgical image guidance system 100 may utilize positional data to track deformations. For example, surgical image guidance system 100 tracking information from an implanted seed to track deformation. Determining/calculating soft tissue deformation is discussed in greater detail below with reference to FIG. 2. In various embodiments, surgical image guidance system 100 provides image guidance to a surgeon during surgery. For example, surgical image guidance system 100 may display a real time 3D volumetric representation of region 154 that accounts for soft tissue deformation caused by the surgeon as they operate.

Referring to FIG. 2, an illustration of a composite display is shown, according to an exemplary embodiment. In various embodiments, the surgical image guidance system of the present disclosure displays a real time position/location of a region/structure of interest (shown as interface 280). Interface 280 may be displayed on a display device for viewing by a care provider (e.g., a surgeon, a nurse, etc.) during surgery. For example, interface 280 may be displayed on a monitor. The surgical image guidance system may generate interface 280 using surface data and/or subsurface data. For example, the surgical image guidance system may generate interface 280 using intraoperative image data (shown as image data 210), preoperative MRI data (shown as MRI data 220), and/or intraoperative ultrasound data (shown as ultrasound data 230).

Image data 210 may include one or more images and/or data derived therefrom of a surface of the region/structure of interest. In various embodiments, image data 210 is captured during surgery (e.g., by surgical image guidance system 100, etc.). MRI data 220 may include one or more MRI images, data derived therefrom, and/or data associated with MRI imaging of a subsurface of the region/structure of interest. In various embodiments, MRI data 220 is captured before surgery. Ultrasound data 230 may include one or more ultrasound images, data derived therefrom, and/or data associated with ultrasound imaging of a subsurface of the region/structure of interest. In various embodiments, ultrasound data 230 is captured during surgery. The surgical image guidance system may align image data 210, MRI data 220, and/or ultrasound data 230 using one or more references (shown as fiducial 212, fiducial 222, anatomy 224 and anatomy 234). For example, the surgical image guidance system may align image data 210 with MRI data 220 using fiducial 212 and fiducial 222. In various embodiments fiducial 212 and fiducial 222 are the same fiducial (or are different fiducials positioned in the same location, etc.). As another example, the surgical image guidance system may align MRI data 220 with ultrasound data 230 using anatomy 224 and anatomy 234. In various embodiments, anatomy 223 and anatomy 234 correspond to the same piece of anatomy. For example, the surgical image guidance system may align MRI data 220 with ultrasound data 230 using a chest wall as a reference. Anatomy 224 and anatomy 234 may be and/or include any sufficiently rigid piece of anatomy to resist soft tissue deformation and serve as a reference. Alignment may include performing image segmentation. For example, the surgical image guidance system may identify (e.g., using landmark extraction, etc.) and segment (e.g., using a deep learning model, etc.) a chest wall in ultrasound data 230 and use the chest wall to align ultrasound data 230 with a 3D volumetric model generated using MRI data 220. In some embodiments, alignment includes colocalizing one or more points. For example, a user may digitize a number of points using a tracked stylus and the surgical image guidance system may use the number of points to determine a relationship between two or more sets of data (e.g., images, models derived from imaging data, etc.).

In various embodiments, the surgical image guidance system uses the surface data (e.g., image data 210) and/or the subsurface data (e.g., MRI data 220 and/or ultrasound data 230) to update a 3D model of at least a portion of a subject's anatomy (shown as model 250). In various embodiments, model 250 is generated using preoperative imaging. For example, model 250 may be generated using preoperative MRI imaging. Additionally or alternatively, model 250 may be generated and/or modified using intraoperative imaging. Model 250 may be and/or include a 3D volumetric model of at least a portion of anatomy. Updating model 250 may include calculating/determining soft tissue deformation. Calculating/determining soft tissue deformation may include optimizing an objective function. For example, the surgical image guidance system may optimize an objective function to iteratively determine an optimal excitation of active control surfaces that serve as boundary conditions in model 250 to resolve measured shape differences. The objective function may include (i) a 3D shape error between a segmented organ surface and a corresponding point cloud representing the segmented organ surface, (ii) a 3D feature error between intraoperative imaging (e.g., ultrasound, etc.) acquired organ subsurface contours and corresponding preoperative imaging (e.g., MRI, etc.) corresponding to the same subsurface contours, (iii) a 3D positional error between preoperative imaging fiducials and their corresponding positions, (iv) a 3D closest point error between imaged features (e.g., falciform and inferior ridges associated with a liver, etc.) and corresponding preoperative imaging acquired features, and/or (v) minimization of a conventional strain energy density term to regularize and avoid overt shape distortion. In various embodiments, the surgical image guidance system generates a 3D volumetric deformation field. The 3D volumetric deformation field may be applied to preoperative imaging to generate a representation of a region/structure of interest in its current deformed state. Additionally or alternatively, the surgical image guidance system may implement an artificial intelligence (AI) algorithm to calculate/determine soft tissue deformation. For example, the surgical image guidance system may generate a point cloud from a segmented surface of a region/structure of interest, encode the point cloud into vector space using a neural network (e.g., a Set Transformer trained via heteroscedastic regression to the solution of the iterative solver and/or via direct minimization of a registration loss via a heteroscedastic loss that minimizes the discrepancy between the deformed model and the observation while predicting an uncertainty value over the deformation field, etc.).

In various embodiments, model 250 is robust to domain invasion (e.g., grid splitting, etc.). For example, the surgical image guidance system may apply an extended finite element method (XFEM) approach to decouple visualization and tissue separation within the optimization calculation, thereby enabling real time deformation calculations including grid-splitting changes (e.g., associated with retraction/resection during surgery, etc.). Additionally or alternatively, model 250 may account nonlinear material behavior caused by heterogeneity and/or anisotropy in the anatomy of a subject. For example, the surgical image guidance system may apply the XFEM approach with a strain hardening effect to account for increased tissue stiffness under retractor compression. As another example, the surgical image guidance system may classify tissue (e.g., as adipose, glandular, lesion, etc.) based on an average voxel intensity as determined from imaging data and assign a parameter to each tissue classification (e.g., a mechanical strain parameter, a deformation vector, etc.).

Interface 280 may facilitate identifying and operating on a region/structure of interest during surgery. For example, interface 280 may display a real time 3D representation of a lesion, may display a real time 3D representation of a surgical instrument, and may update the real time 3D representation of the lesion to account for soft tissue deformation caused by the surgical instrument or other source (e.g., physical interactions, biological changes, etc.) to facilitate a surgeon identifying and removing the lesion. As another example, interface 280 may display a real time 3D representation of vasculature to facilitate a surgeon avoiding the vasculature. In various embodiments, interface 280 includes a visual representation of a region/structure of interest. The visual representation may be a real time representation that accounts for soft tissue deformation. For example, a lesion may deform from a first state (shown as lesion 282) to a second state (shown as lesion 284) that is different from the first state (e.g., has a different shape/structure, etc.) and interface 280 may generate a 3D representation of the lesion in its deformed state.

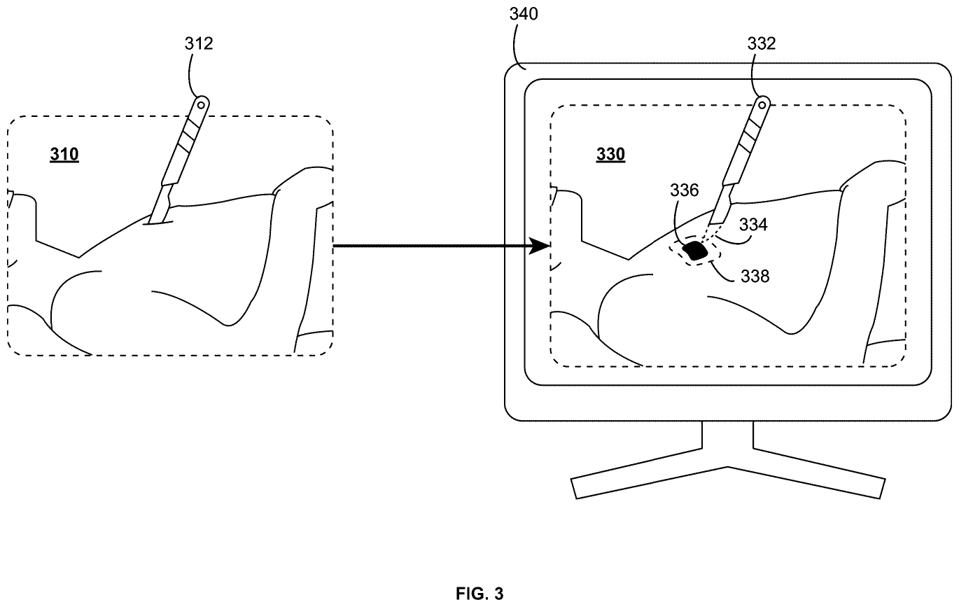

Referring to FIG. 3, an illustration of a surgical guidance display is shown, according to an exemplary embodiment. In various embodiments, the surgical image guidance system tracks one or more surgical instruments and displays a visual representation of the one or more surgical instruments to facilitate a surgical procedure. For example, the surgical image guidance system may track one or more reference markers associated with a scalpel using optical device 110 to generate a visual representation of the scalpel in 3D space.

As shown in FIG. 3, a care provider may use a surgical instrument (shown as scalpel 312) during a procedure (shown as scene 310) and the surgical image guidance system may generate a visual representation of a real time position/orientation of the surgical instrument (shown as scalpel 332) and display the visual representation on a display device (shown as interface 330 displayed on display 340) for viewing by the care provider. Scalpel 332 may include a viewable portion (e.g., a portion of the surgical instrument that is visible by the care provider) and/or an occluded portion (e.g., a portion of the surgical instrument that is not visible by the care provider, etc.) (shown as subsurface portion 334). For example, interface 330 may display a portion of a scalpel that is currently occluded because it is inside the soft tissue of a subject. In various embodiments, interface 330 includes a visual representation of a region/structure of interest (shown as lesion 336). Lesion 336 may be a 3D representation. Additionally or alternatively, interface 330 may include a boundary/volume associated with lesion 336 (shown as margin 338). For example, interface 330 may display a surgical margin for a tumor excision.

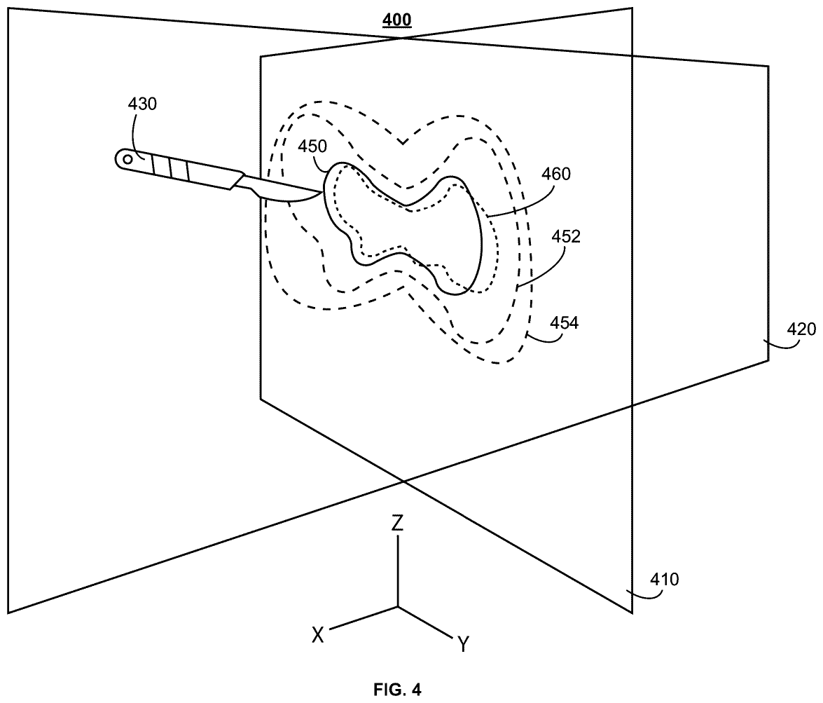

Referring to FIG. 4, an illustration of graphical user interface (GUI) 400 is shown, according to an exemplary embodiment. GUI 400 may be displayed to a care provider (e.g., on display 340, etc.) during surgery to facilitate localization of one or more regions/structures of interest. For example, GUI 400 may facilitate verifying and/or validating a surgical margin. In some embodiments, GUI 400 is and/or includes a 3D volumetric model (e.g., a 3D volumetric model of the anatomy of a subject, etc.). GUI 400 may include one or more imaging planes (shown as first plane 410 and second plane 420). In some embodiments, first plane 410 and/or second plane 420 are and/or include medical imaging data (e.g., an MRI image, an ultrasound image, etc.). For example, first plane 410 and/or second plane 420 may be ultrasound images. As another example, first plane 410 and/or second plane 420 may be real time visualizations of segments of a 3D model of the anatomy of a subject (e.g., as derived from model 250, etc.).

GUI 400 may include a visual representation of one or more surgical instruments (shown as scalpel 430) and/or a visual representation of one or more regions/structures of interest (shown as lesion 460). In various embodiments, a region/structure of interest may deform during surgery. For example, soft tissue including a tumor may deform as a result of the physical pressure of an ultrasound device during intraoperative imaging. GUI 400 may facilitate visualization of a real time position/location of the one or more regions/structures of interest as they deform from an original state (shown as lesion 450) to a deformed state represented by lesion 460. Additionally or alternatively, GUI 400 may include a visual representation of a surgical margin associated with lesion 460 (shown as margin 452). Margin 452 may update in real time according to the deformation of lesion 460. Additionally or alternatively, GUI 400 may include a visual representation of a resection (shown as resection plane 454). The surgical image guidance system may generate resection plane 454 based on tracking a real time position/orientation of a surgical instrument. In various embodiments, resection plane 454 may be used to verify and/or validate compliance with a surgical margin. In some embodiments, GUI 400 includes a modified representation of scalpel 430. For example, GUI 400 may be an undeformed representation of one or more regions/structures of interest and GUI 400 may include a shifted representation of scalpel 430 such that a portion (e.g., a working surface) of scalpel 430 is represented in its deformation-corrected position on the undeformed representation of the one or more regions/structures of interest.

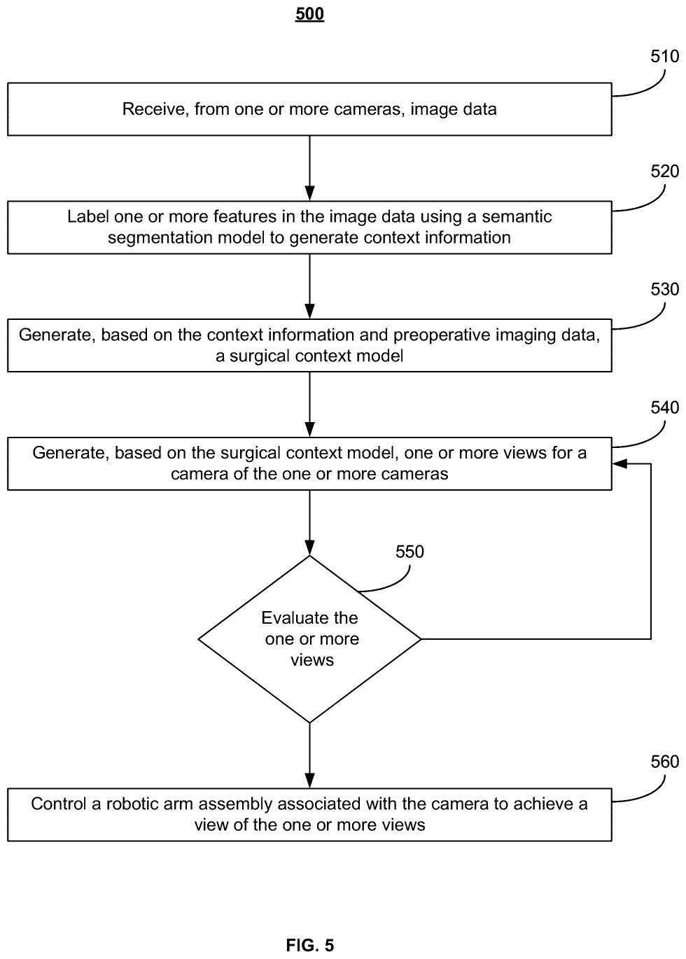

Referring now to FIG. 5, method 500 for positioning a robotic arm assembly is shown, according to an exemplary embodiment. In various embodiments, the surgical image guidance system autonomously controls one or more robotic arm assemblies (e.g., such as robotic arm assemblies 102, etc.). In various embodiments, the surgical image guidance system simultaneously controls a number of robotic arm assemblies to position an optical device and an optical tracking device. For example, the surgical image guidance system may position the optical device such that it has a substantially unobstructed view of a breast surface during a lumpectomy and may position the optical tracking device such that it has line of sight on an intraoperative ultrasound probe and a retractor during the lumpectomy. In some embodiments, the surgical image guidance system positions the optical device by performing constrained optimization. Additionally or alternatively, the surgical image guidance system may position the optical device using an AI model trained and/or executed on data from one or more sources (e.g., MRI data, ultrasound data, images, pose information, etc.). In various embodiments, the surgical image guidance system performs method 500.

At step 510, the surgical image guidance system may receive image data. For example, the surgical image guidance system may receive a number of images from optical device 110. In some embodiments, the surgical image guidance system receives image data from one or more cameras located in a surgical environment (e.g., an operating room, etc.). The image data may be and/or include one or more images. Additionally or alternatively, the image data may be and/or include medical imaging data such as subsurface data generated by intraoperative ultrasound (e.g., one or more images generated by an ultrasound device, etc.). In some embodiments, the image data is and/or includes surface data. For example, the image data may include a point cloud and/or a depth map (e.g., generated from a number of images) that characterizes a surface being operated on (e.g., describes one or more characteristics of the surface such as a 3D layout of the surface, etc.). In some embodiments, the image data includes tracking data. For example, the image data may include tracking data for one or more surgical instruments tracked by optical device 110.

At step 520, the surgical image guidance system may label one or more features in the image data using a semantic segmentation model to generate context information. For example, the surgical image guidance system may segment an image into a number of regions associated with different semantic labels such as object, person, tissue, anatomical structure, anatomical substructure, marker/reference, background, and/or the like. Semantic segmentation may be performed using a deep neural network trained on a corpus of labeled images (e.g., anatomical images, etc.). In some embodiments, semantic segmentation is performed with a segment anything model (SAM). Performing semantic segmentation may include identifying, labeling, and/or tracking various elements such as a surgeons' hands, organ segments, surgical instruments, and/or the like. In some embodiments, step 520 includes generating a point cloud of a surface of a region/structure of interest. For example, the surgical image guidance system may generate a point cloud of a surface of an organ being operated on.

Context information may be and/or include any information describing and/or associated with a surgery. For example, context information may describe a surgical workflow, an entity, an anatomical structure, a positioning of objects and/or people within a space (e.g., an operating room), a condition (e.g., an amount of light, a temperature, a sterility, etc.) of a space, a surgical instrument, and/or the like. As another example, context information may include a point cloud characterizing a surface of an organ being operated on, an image of a surgical scene, a 3D representation of a surgical scene, a position/pose of a surgeon, a current step in a surgical procedure, whether or not a view of an imaging device is occluded, a quality of a field of view of optical device 110, a predicted next step in a surgical workflow, a predicted next position of a nurse in an operating room, types of surgical instrument interactions, the condition of anatomical structure and/or incisions, incision size (e.g., diameter), and/or the like.

At step 530, the surgical image guidance system may generate a surgical context model. In various embodiments, the surgical image guidance system generates the surgical context model based on the context information and/or preoperative imaging data. For example, the surgical image guidance system may generate a surgical context model that describes (i) a 3D position/orientation/pose of a number of care providers, objects, and a subject and their anatomy in an operating room, (ii) a 3D position/orientation/pose of robotic arm assemblies 102 and any associated components such as optical device 110, (iii) a current step in surgical workflow, (iv) predicted next positions of one or more people/objects in a 3D model, and/or the like. As another example, the surgical image guidance system may generate a 3D model of an organ being operated on and may place the 3D model of the organ being operated on into a larger 3D model representing the entire operating room. In various embodiments, the surgical context model is a phantom/virtual representation of an operation room scene. For example, the surgical context model may model a surgical environment including staff, patient anatomy, surgical equipment, and a robotic arm assembly. The phantom/virtual representation may have associated metadata (e.g., context information, etc.). The surgical context model may be used to perform positioning of robotic arm assemblies 102. For example, the surgical image guidance system may analyze the surgical context model to determine a position of a robotic arm assembly with respect to members of a surgical team and anatomy of a subject being operated on.

At step 540, the surgical image guidance system may generate one or more views for a camera of the one or more cameras. For example, the surgical image guidance system may generate a view within a 3D model of the surgical context model that allows optical device 110 to view an organ being operated on. In various embodiments, the surgical image guidance system generates the one or more views based on the surgical context model. For example, the surgical image guidance system may generate a view using model predictive control and/or a neural network (e.g., ScoreNet, etc.). Generating the one or more views may include determining adjustments to one or more robotic arm assemblies to achieve the one or more views. In various embodiments, the one or more views include a region/structure of interest. For example, the one or more views may be views of a lesion being operated on. In some embodiments, the one or more views are generated to facilitate tracking fiducials, surgical instruments, physical anatomy, and/or the like. For example, a view may be generated that includes a surgical instrument used by a surgeon.

At step 550, the surgical image guidance system may evaluate the one or more views. In various embodiments, evaluating the one or more views includes scoring the one or more views. For example, each view may be scored according to how well the view provides an unobstructed view of an organ being operated on, how much a robotic arm assembly has to obstruct other people and/or objects in a surgical scene to achieve the view, how much a robotic arm assembly has to move to achieve the view, whether the view is possible (e.g., whether a robotic arm assembly would have to travel through space currently occupied by a person and/or object to achieve the view, etc.), how close a robotic arm assembly would have to be to a person in order to achieve the view, the likelihood that a position required of a robotic arm assembly to achieve the view is a position that a person may be likely to occupy in the near future, whether the view will still be relevant during a next step in a surgery workflow, whether the view requires a robotic arm assembly to block surgery lighting, and/or the like. In some embodiments, step 550 includes comparing the one or more scores to a threshold. If a view of the one or more views satisfies the evaluation criteria, then method 500 may continue at step 560. If a view of the one or more views does not satisfy the evaluation criteria, then method 500 may continue at step 540. In some embodiments, step 550 includes selecting a view having a highest score. Additionally or alternatively, step 550 may include selecting a view that satisfies a threshold (e.g., exceeds a threshold, is below a threshold, etc.). In some embodiments, step 550 includes selecting a view that optimizes information gained about a patient anatomy by reducing uncertainty.

At step 560, the surgical image guidance system may control a robotic arm assembly associated with the camera to achieve a view of the one or more views. In various embodiments, step 560 includes controlling one or more degrees of freedom of the robotic arm assembly. For example, step 560 may include operating the robotic arm assembly to control the robotic arm assembly in six degrees of freedom using ray casting. In various embodiments, the surgical image guidance system controls the robotic arm assembly to achieve a view selected in step 550.

In various embodiments, method 500 includes controlling a robotic arm assembly according to a safety-aware manipulation framework. For example, the surgical image guidance system may receive inputs from a sensory skin of the robotic arm assembly and/or ceiling mounted cameras in an operating room and perform a safety monitoring process that provides in situ surgical situational awareness of the robotic arm assembly and facilitate motion control thereof to achieve a next-best view for and RDBd scanner and/or substantial marker visibility measures for an optical tracker.

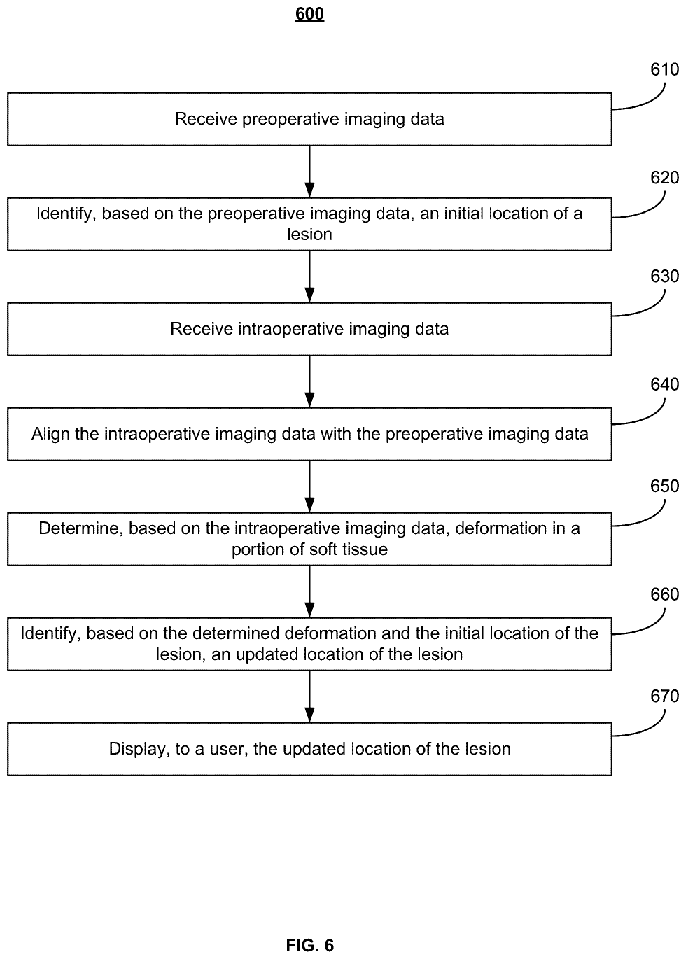

Referring now to FIG. 6, method 600 for surgical image guidance is shown, according to an exemplary embodiment. In various embodiments, the surgical image guidance system performs method 600. Method 600 may facilitate localizing and removing a lesion in deformable soft tissue. For example, the surgical image guidance system may track a 3D representation of a tumor as it deforms during surgery, may display the 3D representation to a surgeon, and the surgeon may view the 3D representation to excise the tumor.

At step 610, the surgical image guidance system may receive preoperative imaging data. For example, the surgical image guidance system may receive MRI data. The preoperative imaging data may be and/or include images and/or information derived from images. For example, the preoperative imaging data may include a 3D model of anatomy of a subject generated based on one or more MRI images. In some embodiments, the preoperative imaging data includes highlighted portions. For example, the preoperative imaging data may include ink-on-tumor contrasting to facilitate identification of a tumor.

At step 620, the surgical image guidance system may identify an initial location of a lesion. For example, the surgical image guidance system may perform feature extraction on one or more images to identify a region in the one or more images corresponding to a lesion. In various embodiments, the surgical image guidance system identifies the initial location of the lesion based on the preoperative imaging data. For example, the surgical image guidance system may analyze the preoperative imaging data using a machine learning model trained on a corpus of medical imaging data with diseased areas labeled. In some embodiments, step 620 includes identifying a 3D volume that corresponds to the lesion. For example, the surgical image guidance system may generate a 3D volumetric model of anatomy of a patient based on a number of MRI images and may identify a portion of the 3D volumetric model as representing the lesion. In some embodiments, step 620 includes identifying a highlighted portion within MRI data. For example, the surgical image guidance system may identify a tumor based on the presence of a contrast dye in one or more MRI images.

At step 630, the surgical image guidance system may receive intraoperative imaging data. For example, the surgical image guidance system may receive one or more ultrasound images. Intraoperative imaging data may be and/or include data from an ultrasound device, data from an optical device (e.g., optical device 110), data from an optical tracking device (e.g., optical tracking device 112, etc.), and/or the like. The intraoperative imaging data may be and/or include surface data and/or subsurface data. In various embodiments, the intraoperative imaging data includes one or more images and/or information derived therefrom. In some embodiments, the intraoperative imaging data includes position/location data. For example, the surgical image guidance system may receive images from a tracked ultrasound device and tracking information describing a 3D position of the tracked ultrasound device.

At step 640, the surgical image guidance system may align the intraoperative imaging data with the preoperative imaging data. For example, the surgical image guidance system may align the intraoperative imaging data with the preoperative imaging data using one or more fiducial and/or references. Alignment is discussed in greater detail above with reference to FIG. 2. In various embodiments, step 640 includes generating a composite. Step 640 may include aligning one or more 3D models.

At step 650, the surgical image guidance system may determine deformation in a portion of the soft tissue. For example, the surgical image guidance system may generate a volumetric 3D deformation field. In various embodiments, the surgical image guidance system determines deformation in the portion of the soft tissue based on the intraoperative imaging data. For example, the surgical image guidance system may identify a change in a structure of a tumor based on one or more ultrasound images. Additionally or alternatively, the surgical image guidance system may determine deformation based on the preoperative imaging data. For example, the surgical image guidance system may compare a location of a tumor in one or more MRI images to a location of the tumor in one or more ultrasound images. Determining deformation is discussed in greater detail above with reference to FIG. 2.

At step 660, the surgical image guidance system may identify an updated location of the lesion. For example, the surgical image guidance system may update a 3D model of the lesion to reflect a real time position/orientation of the lesion. In various embodiments, the surgical image guidance system identifies the updated location of the lesion based on the determined deformation and/or the initial location of the lesion. For example, the surgical image guidance system may update the initial location represented by a first 3D model by applying a 3D volumetric deformation field to the first 3D model to generate a second 3D model representing the updated location. In various embodiments, the updated location is a location in 3D space. For example, the updated location may be a 3D model positioned in 3D space.

At step 670, the surgical image guidance system may display the updated location of the lesion to a user. For example, the surgical image guidance system may cause an interface to be displayed on a display device. In various embodiments, displaying the updated location of the lesion includes displaying a 3D model of the lesion. Additionally or alternatively, step 670 may include overlaying one or more elements onto the display. For example, the surgical image guidance system may overlay a 3D representation of a surgical instrument on the display to facilitate locating the surgical instrument relative to the 3D model of the lesion.

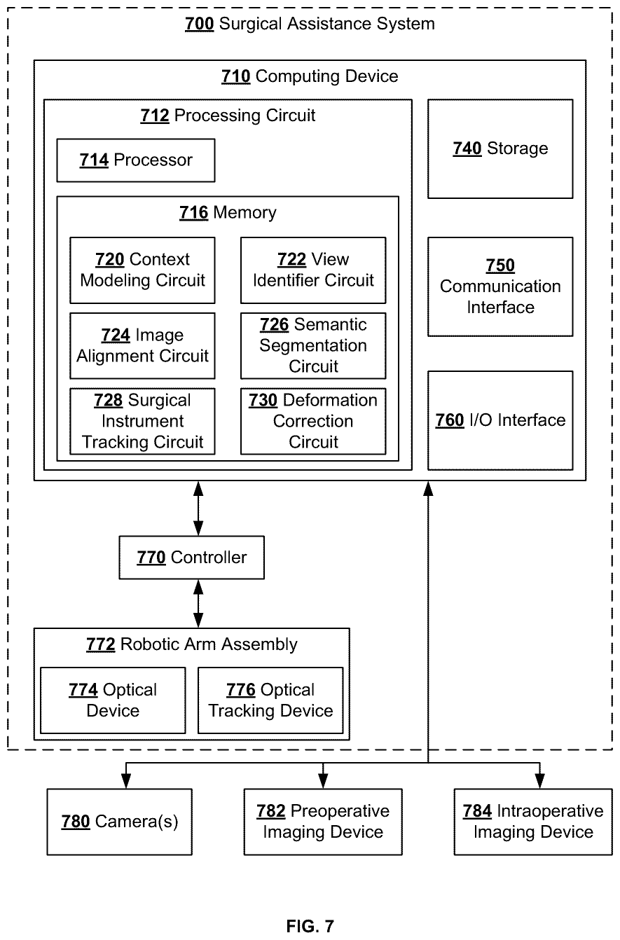

Referring now to FIG. 7, surgical assistance system 700 is shown, according to an exemplary embodiment. Surgical assistance system 700 may be and/or include surgical image guidance system 100. In various embodiments, surgical assistance system 700 facilitates surgical image guidance to determine a location of a region of interest during surgery and display the location to a care provider (e.g., surgeon, nurse, etc.). Surgical assistance system 700 may reduce reoperation rates, reduce the length of operation, and/or increase surgical accuracy. Surgical assistance system 700 is shown to include computing device 710, controller 770, and robotic arm assembly 772. Computing device 710 may be and/or include any computing device. For example, computing device 710 may be a desktop computer including application-specific hardware and/or software or a server. In various embodiments, computing device 710 communicates with robotic arm assembly 772 via controller 770. Controller 770 may include hardware and/or software to facilitate operation of robotic arm assembly 772. For example, computing device 710 may determine a view for a camera, may determine control signals to cause robotic arm assembly 772 to move to achieve the view, may send the control signals to controller 770, and controller 770 may generate low-level instructions for robotic arm assembly 772 to cause robotic arm assembly to move in accordance with the control signals. Computing device 710 and controller 770 may control robotic arm assembly 772 autonomously (e.g., with little to no input from a user). For example, computing device 710 may continuously position robotic arm assembly 772 such that optical device 774 has a view of an area being operated on during a surgery (e.g., repositioning/accounting for obstructions, etc.). Robotic arm assembly 772 may be and/or include robotic arm assemblies 102. Robotic arm assembly 772 may include one or more articulating components and control mechanisms for controlling the one or more articulating components. In various embodiments, robotic arm assembly 772 is capable of moving in two or more degrees of freedom. For example, robotic arm assembly 772 may be capable of moving in six degrees of freedom.

Computing device 710 may include processing circuit 712, storage 740, communication interface 750, storage 740, and/or I/O interface 760. Processing circuit 712 may include processor 714 and/or memory 716. Processor 714 may be a general purpose or specific purpose processor, an application specific integrated circuit (ASIC), one or more field programmable gate arrays (FPGAs), a group of processing components, or other suitable processing components. Processor 714 is configured to execute computer code or instructions stored in memory 716 or received from other computer readable media (e.g., CDROM, network storage, a remote server, etc.). Memory 716 may include one or more devices (e.g., memory units, memory devices, storage devices, and/or other computer-readable media) for storing data and/or computer code for completing and/or facilitating the various processes described in the present disclosure. Memory 716 may include random access memory (RAM), read-only memory (ROM), hard drive storage, temporary storage, non-volatile memory, flash memory, optical memory, or any other suitable memory for storing software objects and/or computer instructions. Memory 716 may include database components, object code components, script components, or any other type of information structure for supporting the various activities and information structures described in the present disclosure. Memory 716 may be communicably connected to processor 714 via processing circuit 712 and may include computer code for executing (e.g., by processor 714) one or more of the processes described herein. For example, memory 716 may have instructions stored thereon that, when executed by processor 714, cause processing circuit 712 to (i) receive surface data characterizing a portion of soft tissue, (ii) receive subsurface data characterizing a subsurface of the portion of soft tissue, (iii) measure, based on the surface data, deformation of the surface of the portion of soft tissue, (iv) update, based on the deformation of the surface and the subsurface data, a model including a representation of a region of interest, and (v) transmit a visual representation of the model to be displayed to a user.

Memory 716 is shown to include context modeling circuit 720, view identifier circuit 722, image alignment circuit 724, semantic segmentation circuit 726, surgical instrument tracking circuit 728, and deformation correction circuit 730. Context modeling circuit 720 may generate a surgical context model. In various embodiments, context modeling circuit 720 generates the surgical context model by analyzing one or more images. For example, context modeling circuit 720 may receive one or more images from optical device 774 and/or camera(s) 780 and may generate a model of a surgical scene to understand where people and objects in the surgical scene are positioned and what step in a surgical workflow is currently being performed. In various embodiments, context modeling circuit 720 generates the surgical context model based on context information and/or preoperative imaging data. For example, context modeling circuit 720 may generate a surgical context model that describes (i) a 3D position/orientation/pose of a number of care providers, objects, and a subject and their anatomy in an operating room, (ii) a 3D position/orientation/pose of robotic arm assemblies 102 and any associated components such as optical device 110, (iii) a current step in surgical workflow, (iv) predicted next positions of one or more people/objects in a 3D model, and/or the like. In some embodiments, the surgical context model is and/or includes model 250.

View identifier circuit 722 may identify one or more views for cameras to observe an operating room and/or objects/people within the operating room. In some embodiments, view identifier circuit 722 performs one or more steps of method 500. View identifier circuit 722 may generate potential next views, score each of the potential next views, and/or select a view of the potential next views based on the scores. In various embodiments, view identifier circuit 722 identifies views using a neural network such as ScoreNet. In various embodiments, view identifier circuit 722 controls movement of robotic arm assembly 772 (e.g., via controller 770, etc.).

Image alignment circuit 724 aligns one or more images and/or one or more sets of image data. For example, image alignment circuit 724 may align image data from as described with reference to FIG. 2 above. In various embodiments, image alignment circuit 724 uses one or more references/fiducials to align image data. For example, image alignment circuit 724 may align preoperative MRI images with intraoperative ultrasound images and/or intraoperative photos using in situ fiducials.

Semantic segmentation circuit 726 may perform semantic segmentation on one or more images and/or image data. For example, semantic segmentation circuit 726 may identify and label/classify one or more features of an image such as objects, people, organs, portions of organs, and/or the like. In some embodiments, semantic segmentation circuit 726 implements a deep learning model. For example, semantic segmentation circuit 726 may implement a transformer-based architecture using multi-head self-attention and/or convolutional layers. As another example, semantic segmentation circuit 726 may implement a U-Netr, TransUnet, and/or SAM algorithm.

Surgical instrument tracking circuit 728 may track one or more surgical instruments. For example, surgical instrument tracking circuit 728 may receive tracking data from optical tracking device 776 and use the tracking data to track a surgical instrument such as a scalpel. In various embodiments, surgical instrument tracking circuit 728 tracks one or more surgical instruments using image recognition. For example, surgical instrument tracking circuit 728 may track a position/location/orientation of a surgical instrument across one or more images/frames by identifying one or more features of the surgical instrument. Additionally or alternatively, surgical instrument tracking circuit 728 may track the one or more surgical instruments using one or more references. For example, surgical instrument tracking circuit 728 may track a surgical instrument using a number of reflective markers associated with the surgical instrument. In some embodiments, surgical instrument tracking circuit 728 tracks objects in 3D. For example, surgical instrument tracking circuit 728 may determine timeseries data describing a position/orientation of an ablation device in 3D space. In some embodiments, surgical instrument tracking circuit 728 tracks an ultrasound probe.

Deformation correction circuit 730 may determine soft tissue deformation. In some embodiments, deformation correction circuit 730 generates a visualization of tissue deformation. For example, deformation correction circuit 730 may update a 3D model of a lesion to reflect deformation caused by surgical interactions. As another example, deformation correction circuit 730 may generate GUI 400. Deformation correction circuit 730 may perform one or more steps of method 600. In various embodiments, deformation correction circuit 730 performs deformation correction using aligned images from image alignment circuit 724. In various embodiments, deformation correction circuit 730 generates a volumetric 3D deformation field. Deformation correction circuit 730 may apply the volumetric 3D deformation field to image data and/or a 3D model to generate a visualization of deformed soft tissue. For example, deformation correction circuit 730 may generate a visualization of a lesion deformed during surgery by adjusting one or more ultrasound images in real time.

Communication interface 750 may facilitate communication with one or more systems/devices. For example, computing device 710 may communicate via communication interface 750 with a database storing a corpus of labeled imaging data for use in training one or more AI models. As another example, computing device 710 may communicate via communication interface 750 with camera(s) 780. As another example, computing device 710 may communicate via communication interface 750 with preoperative imaging device 782. As another example, computing device 710 may communication via communication interface 750 with intraoperative imaging device 784. Communication interface 750 may be or include wired or wireless communications interfaces (e.g., jacks, antennas, transmitters, receivers, transceivers, wire terminals, etc.) for conducting data communications with external systems or devices. In various embodiments, communications via communication interface 750 is direct (e.g., local wired or wireless communications). Additionally or alternatively, communications via communication interface 750 may utilize a network (e.g., a WAN, the Internet, a cellular network, etc.).

Storage 740 may store data/information associated with the various methods/operations described herein. For example, storage 740 may store a 3D model of anatomy of a patient (e.g., model 250, etc.). As another example, storage 740 may store MRI images, ultrasound images, and/or images from camera(s) 780. Storage 740 may be and/or include one or more memory devices (e.g., hard drive storage, temporary storage, non-volatile memory, flash memory, optical memory, and/or any other suitable memory device).

I/O interface 760 may facilitate input/output operations. For example, I/O interface 760 may include a display capable of presenting information from a user and an interface capable of receiving input from the user. In some embodiments, I/O interface 760 includes a display device configured to present a GUI to a user. I/O interface 760 may include hardware and/or software components. For example, I/O interface 760 may include a physical input device (e.g., a mouse, a keyboard, a touchscreen device, etc.) and software to enable the physical input device to communicate with computing device 710 (e.g., firmware, drivers, etc.). In some embodiments, I/O interface 760 includes an API to facilitate interaction with external systems (e.g., an augmented reality display system, etc.).

Robotic arm assembly 772 may include optical device 774 and/or optical tracking device 776. Optical device 774 may include any instrument capable of capturing and/or storing images and/or video. In various embodiments, optical device 774 is a camera. For example, optical device 774 may be a stereo camera. Additionally or alternatively, optical device 774 may include a fiber optic sensor, a photodiode/photodetector, a light detection and ranging device, an infrared camera, a time-of-flight sensor, a structured light scanner, a laser rangefinder, an optical coherence tomography device, a microscope, and/or the like. Optical device 774 may be the same as optical device 110. Optical tracking device 776 may include an optical device and/or software configured to identify and track a person and/or object. For example, optical tracking device 776 may include a camera and software for tracking one or more surgical instruments. In some embodiments, optical tracking device 776 uses one or more reference markers (e.g., reflective markers coupled to a surgical instrument, fiducials, etc.). Optical tracking device 776 may be the same as optical tracking device 112.

Camera(s) 780 may include any instrument capable of capturing and/or storing images and/or video. Camera(s) 780 may be and/or include optical device 110. In various embodiments, camera(s) 780 include one or more cameras positioned in an operating room. Camera(s) 780 may have a field of view such that they may observe care providers during surgery. Preoperative imaging device 782 may be and/or include any imaging device. For example, preoperative imaging device 782 may include an MRI system, an ultrasound system, an x-ray system, a computed tomography (CT) system, an elastography imaging system, an echocardiography imaging system, a spectroscopy imaging system (e.g., near-infrared spectroscopy system, etc.), a magnetic particle imaging (MPI) system, and/or the like. In various embodiments, preoperative imaging device 782 captures image data of anatomy of a subject prior to a surgery. For example, preoperative imaging device 782 may be an MRI system that is used to generate an image of anatomy of a subject prior to surgery.

Intraoperative imaging device 784 may be and/or include any imaging device. For example, intraoperative imaging device 784 may include an MRI system, an ultrasound system, an x-ray system, a computed tomography (CT) system, an elastography imaging system, an echocardiography imaging system, a spectroscopy imaging system (e.g., near-infrared spectroscopy system, etc.), a magnetic particle imaging (MPI) system, and/or the like. In various embodiments, intraoperative imaging device 784 captures image data of anatomy of a subject during surgery. For example, intraoperative imaging device 784 may be an ultrasound system that is used to locate a surgical instrument relative to anatomy of a subject during surgery. Intraoperative imaging device 784 may be the same as ultrasound device 130.

As utilized herein with respect to numerical ranges, the terms “approximately,” “about,” “substantially,” and similar terms generally mean+/−10% of the disclosed values, unless specified otherwise. As utilized herein with respect to structural features (e.g., to describe shape, size, orientation, direction, relative position, etc.), the terms “approximately,” “about,” “substantially,” and similar terms are meant to cover minor variations in structure that may result from, for example, the manufacturing or assembly process and are intended to have a broad meaning in harmony with the common and accepted usage by those of ordinary skill in the art to which the subject matter of this disclosure pertains. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

References herein to the positions of elements (e.g., “top,” “bottom,” “above,” “below”) are merely used to describe the orientation of various elements in the figures. It should be noted that the orientation of various elements may differ according to other exemplary embodiments, and that such variations are intended to be encompassed by the present disclosure.

The present disclosure contemplates methods, systems, and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general-purpose computer, special purpose computer, or special purpose processing machines to perform a certain function or group of functions.

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

The term “client or “server” include all kinds of apparatus, devices, and machines for processing data, including by way of example a programmable processor, a computer, a system on a chip, or multiple ones, or combinations, of the foregoing. The apparatus may include special purpose logic circuitry, e.g., a field programmable gate array (FPGA) or an application specific integrated circuit (ASIC). The apparatus may also include, in addition to hardware, code that creates an execution environment for the computer program in question (e.g., code that constitutes processor firmware, a protocol stack, a database management system, an operating system, a cross-platform runtime environment, a virtual machine, or a combination of one or more of them). The apparatus and execution environment may realize various different computing model infrastructures, such as web services, distributed computing and grid computing infrastructures.

The systems and methods of the present disclosure may be completed by any computer program. A computer program (also known as a program, software, software application, script, or code) may be written in any form of programming language, including compiled or interpreted languages, declarative or procedural languages, and it may be deployed in any form, including as a stand-alone program or as a module, component, subroutine, object, or other unit suitable for use in a computing environment. A computer program may, but need not, correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one computer or on multiple computers that are located at one site or distributed across multiple sites and interconnected by a communication network.

The processes and logic flows described in this specification may be performed by one or more programmable processors executing one or more computer programs to perform actions by operating on input data and generating output. The processes and logic flows may also be performed by, and apparatus may also be implemented as, special purpose logic circuitry (e.g., an FPGA or an ASIC).

Processors suitable for the execution of a computer program include, by way of example, both general and special purpose microprocessors, and any one or more processors of any kind of digital computer. Generally, a processor will receive instructions and data from a read only memory or a random-access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data (e.g., magnetic, magneto-optical disks, or optical disks). However, a computer need not have such devices. Moreover, a computer may be embedded in another device (e.g., a vehicle, a Global Positioning System (GPS) receiver, etc.). Devices suitable for storing computer program instructions and data include all forms of non-volatile memory, media and memory devices, including by way of example semiconductor memory devices (e.g., EPROM, EEPROM, and flash memory devices; magnetic disks, e.g., internal hard disks or removable disks; magneto-optical disks; and CD ROM and DVD-ROM disks). The processor and the memory may be supplemented by, or incorporated in, special purpose logic circuitry.

To provide for interaction with a user, implementations of the subject matter described in this specification may be implemented on a computer having a display device (e.g., a CRT (cathode ray tube), LCD (liquid crystal display), OLED (organic light emitting diode), TFT (thin-film transistor), or other flexible configuration, or any other monitor for displaying information to the user. Other kinds of devices may be used to provide for interaction with a user as well; for example, feedback provided to the user may be any form of sensory feedback (e.g., visual feedback, auditory feedback, or tactile feedback).

Implementations of the subject matter described in this disclosure may be implemented in a computing system that includes a back-end component (e.g., as a data server), or that includes a middleware component (e.g., an application server), or that includes a front end component (e.g., a client computer) having a graphical user interface or a web browser through which a user may interact with an implementation of the subject matter described in this disclosure, or any combination of one or more such back end, middleware, or front end components. The components of the system may be interconnected by any form or medium of digital data communication (e.g., a communication network). Examples of communication networks include a LAN and a WAN, an inter-network (e.g., the Internet), and peer-to-peer networks (e.g., ad hoc peer-to-peer networks).

What is claimed is:

1. A surgical image guidance system, comprising:
a processing circuit comprising a processor and memory, the memory having instructions stored thereon that, when executed by the processor, cause the processing circuit to:
receive, from an optical device, surface data characterizing a surface of a portion of soft tissue;
receive, from an intraoperative imaging device, subsurface data characterizing a subsurface of the portion of soft tissue, wherein the subsurface comprises a region of interest;
measure, based on the surface data, deformation of the surface of the portion of soft tissue;
update, based on at least one of (i) the deformation of the surface and (ii) the subsurface data, a model comprising a representation of the region of interest; and
transmit a visual representation of the model to be displayed to a user.

2. The surgical image guidance system of claim 1, wherein the intraoperative imaging device comprises an ultrasound image device.

3. The surgical image guidance system of claim 1, wherein the optical device comprises a camera.

4. The surgical image guidance system of claim 3, wherein the camera comprises a stereo camera.

5. The surgical image guidance system of claim 1, wherein the optical device comprises an optical tracker.

6. The surgical image guidance system of claim 1, wherein the instructions further cause the processing circuit to:
track a position of a surgical instrument; and
display the position of the surgical instrument on the visual representation of the model.

7. The surgical image guidance system of claim 1, wherein the region of interest comprises soft tissue to be removed, and wherein the visual representation of the model comprises an illustration of a surgical margin associated with the soft tissue to be removed.

8. The surgical image guidance system of claim 1, wherein updating the model comprises updating the representation of the region of interest to reflect deformation of the subsurface of the portion of soft tissue.

9. The surgical image guidance system of claim 1, wherein the instructions further cause the processing circuit to:
receive imaging data characterizing the subsurface of the of the portion of soft tissue; and
generate, based on the imaging data, the model comprising the representation of the region of interest.

10. The surgical image guidance system of claim 9, wherein the imaging data comprises magnetic resonance imaging (MRI) data.

11. A surgical assistance system, comprising:
a robotic arm assembly having an optical device coupled thereto; and
a processing circuit comprising a processor and memory, the memory having instructions stored thereon that, when executed by the processor, cause the processing circuit to:
autonomously control movement of the robotic arm assembly to position the optical device such that the optical device has at least a partial view of (i) a portion of soft tissue under operation or (ii) a fiducial associated with the portion of soft tissue;
receive, from the optical device, surface data characterizing a surface of the portion of soft tissue under operation;
determine, based on the (i) surface data and (ii) intraoperative ultrasound data, deformation of a subsurface of the portion of soft tissue under operation; and
cause a visual representation of the deformation of the subsurface to be displayed to a user; and
wherein the visual representation comprises an indication of (i) diseased soft tissue or (ii) a region of interest.

12. The surgical assistance system of claim 11, wherein the instructions further cause the processing circuit to:
track a position of at least one of (i) an object or (ii) an entity in 3D space; and
wherein autonomously controlling movement of the robotic arm assembly comprises controlling movement of the robotic arm assembly based on the position of the object or entity in 3D space.

13. The surgical assistance system of claim 11, wherein the optical device comprises at least one of an optical tracking device or a stereo camera.

14. The surgical assistance system of claim 11, wherein determining deformation of the subsurface comprises updating a model of the subsurface generated based on magnetic resonance imaging (MRI) data.

15. A method of providing surgical image guidance, comprising:
receiving preoperative imaging data of at least a portion of a subject;
identifying, based on the preoperative imaging data, an initial location of a lesion;
receiving intraoperative imaging data of the portion of the subject;
identifying a subsequent location of the lesion by determining, based on the intraoperative imaging data, soft tissue deformation of the portion of the subject; and
displaying, to a user, the subsequent location of the lesion.

16. The method of claim 15, wherein determining soft tissue deformation comprises:
aligning the preoperative imaging data with the intraoperative imaging data using a fiducial to form a composite; and
updating a 3D volumetric model of the portion of the subject based on the composite.

17. The method of claim 15, wherein the intraoperative imaging data comprises at least one of (i) image data from a stereo camera or (ii) subsurface data from an ultrasound device.

18. The method of claim 15, wherein the method further comprises:
tracking a position of a surgical instrument; and
wherein displaying the subsequent location of the lesion comprises displaying the position of the surgical instrument relative to the subsequent location of the lesion.

19. The method of claim 18, wherein displaying the subsequent location of the lesion comprises displaying a 3D model of the lesion and a visual representation of the surgical instrument.

20. The method of claim 15, wherein the method further comprises controlling a position of a robotic arm assembly based on the intraoperative imaging data, and wherein controlling the position of the robotic arm assembly comprises adjusting a field of view of an imaging device such that the field of view at least partially includes the portion of the subject.
